(19) United States
(12) Patent Application Publication
(10) Pub. No.: US 2018/0339298 A1
(43) Pub. Date: Nov. 29, 2018

(54) APPARATUS FOR PROCESSING CANNABIS

(71) Applicant: Race Mayers, Kailua-Kona, HI (US)

(72) Inventor: Race Mayers, Kailua-Kona, HI (US)

(21) Appl. No.: 15/801,103

(22) Filed: Nov. 1, 2017

Related U.S. Application Data
(60) Provisional application No. 62/510,903, filed on May 25, 2017.

(51) Int. Cl. B02C 23/10; B03B 4/02; G01G 19/52; G01G 19/40; A61K 36/185

(52) U.S. Cl. CPC B02C 23/10; B03B 4/02; G01G 19/52; B07B 2201/02; A61K 36/185; A61K 2236/51; A61K 2236/15; G01G 19/40

(57) ABSTRACT

An apparatus for processing cannabis plants includes a support-frame, a hopper, a filter-tray, an oscillation means, a weighing station and a power supply. The apparatus for processing cannabis plants is useful for processing, weighing and packaging cannabis after the cannabis has flowered and is harvested.

### CROSS REFERENCE TO RELATED APPLICATION

The present application is related to and claims priority to U.S. Provisional Patent Application No. 62/510,903 filed May 25, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of cannabis harvesting and more specifically relates to an apparatus for processing, weighing and packaging cannabis after harvest.

### 2. Description of Related Art

With the advent of legalizing the medicinal use of cannabis in 16 states the extraction of medicinal compounds from cannabis plant material in large volumes is in demand The most significant driver for this demand are the increasing bodies of research showing tangible benefits to individuals suffering from a plurality of ailments when using medicinal cannabis. Once the cannabis is harvested and dried, the flower must be trimmed, duffed, weighed, and packaged. Duffing is typically done by hand, which can require five laborers and five minutes per pound. In addition, when done by hand, the oscillation speed may be inconsistent and the output may vary. Thus, a suitable solution is desired.

U.S. Pub. No. 2017/0001200 to Mitchell Leffel relates to a cannabis plant filter and apparatus. The described cannabis plant filter and apparatus includes an assembly for separating cannabis trichomes by a vibrating platform and at least one filter.

### BRIEF SUMMARY OF THE INVENTION

In view of the foregoing disadvantages inherent in the known cannabis harvesting art, the present disclosure provides a novel apparatus for processing cannabis. The general purpose of the present disclosure, which will be described subsequently in greater detail, is to provide an apparatus for stripping leaves and stems from cannabis plants and for weighing and packaging the cannabis via the described apparatus.

An apparatus for processing cannabis plants is disclosed herein. The apparatus for processing cannabis plants may include a support-frame including a hopper having an inner-volume defined by at least four-side walls (in preferred embodiments—other arrangements for the hopper sidewalls may be used), an open-top and a sloped-bottom relative to a floor-surface. The inner-volume may be configured to receive at least portions of the cannabis plants, and the sloped-bottom may include an opening configured to discharge the cannabis plants therethrough.

Further, the support-frame may include a filter-tray which may include a downward-slope, at least two support rails, and a filter-screen base. The downward-slope may have an upward-end and a downward-end. Further, the at least two support-rails may be configured to support the filter-tray thereon, and the filter-tray may be configured to receive the cannabis plants discharged from the opening of the hopper.

Additionally, an oscillation means may be connected to the filter-tray and may be configured to oscillate the filter-tray in a forward and backward direction. In a preferred embodiment, the oscillation in the forward and backward direction may be configured to strip the cannabis plants of the plurality of leaves and stems, and the oscillation in the forward and backward direction may be further configured to force the plurality of leaves and stems through the filter-screen base of the filter-tray. In addition to this, the oscillation in the forward and backward direction and the downward-slope may be configured to direct the stripped cannabis plants toward the downward-end of the filter-tray.

The support-frame may also include a weighing station attached to the downward-end of the filter-tray, and the weighing station may include a weighing means. The weighing station may be configured to receive the stripped cannabis, and the weighing means may be configured to weigh the stripped cannabis. A power supply may also be provided which may be configured to supply power to the oscillation means.

A method of using an apparatus for processing cannabis plants is also disclosed herein. The method of using an apparatus for processing cannabis plants may comprise the steps of: providing the apparatus as above; switching on the power supply to the oscillation means; loading the cannabis plants into the inner-volume of the hopper; recording a weight of the stripped cannabis atop the weighing means; packaging the stripped cannabis; and switching off the power supply to the oscillation means.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, an apparatus for processing cannabis, constructed and operative according to the teachings of the present disclosure.

FIG. 1 is a side perspective view of the apparatus for processing cannabis plants, according to an embodiment of the disclosure.

FIG. 2 is a front perspective showing a close-up view of the apparatus for processing cannabis plants according to an embodiment of the present disclosure.

FIG. 3 is a rear perspective view of the apparatus for processing cannabis plants of FIG. 1, according to an embodiment of the present disclosure.

FIG. 4 is an aerial perspective view of the inside of a hopper of the apparatus for processing cannabis plants according to an embodiment of the present disclosure.

FIG. 5 is a front perspective view of the apparatus for processing cannabis plants of FIG. 1, according to an embodiment of the present disclosure.

FIG. 6 is a flow diagram illustrating a method of use for the apparatus for processing cannabis plants, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to cannabis processing and more particularly to an apparatus for processing cannabis as used to improve the process for stripping, weighing and packaging cannabis.

Generally, disclosed is an electric powered mechanical machine used to separate leaves and stems from the cannabis flower via oscillation. It may offer a way to reduce the time and labor spent by cannabis cultivators separating leaves and stems. The machine may create a sifting device for duffing marijuana that operates at a consistent speed. This allows for the weighing of cannabis and the packaging. The present disclosure may enable users to duff one pound of cannabis in one minute using two laborers.

The machine may be comprised of a hopper constructed preferably from molded fiberglass and contains the welded aluminum auger that feeds into the shaker box. A variable speed motor may be connected to the shaker box and controls the auger and shaker box. The bottom of the shaker box may be a screen that allows for leaf and stem to fall through during oscillation. The shaker box may be mounted on rails that allow for the box to oscillate forward and backwards. In one embodiment, the rails and shaker box may be constructed of welded aluminum. The screen may be constructed of punched aluminum sheet metal. The rails may be mounted on stainless steel-all thread, which may allow the angle of the rails/shaker box to be adjusted as desired. At the end of the shaker box opposite of the hopper is where the ‘finished’ cannabis may be ejected and funneled into a container. The container may sit atop a digital scale. The machine may be operated by an on/off pedal that is connected to the variable speed motor.

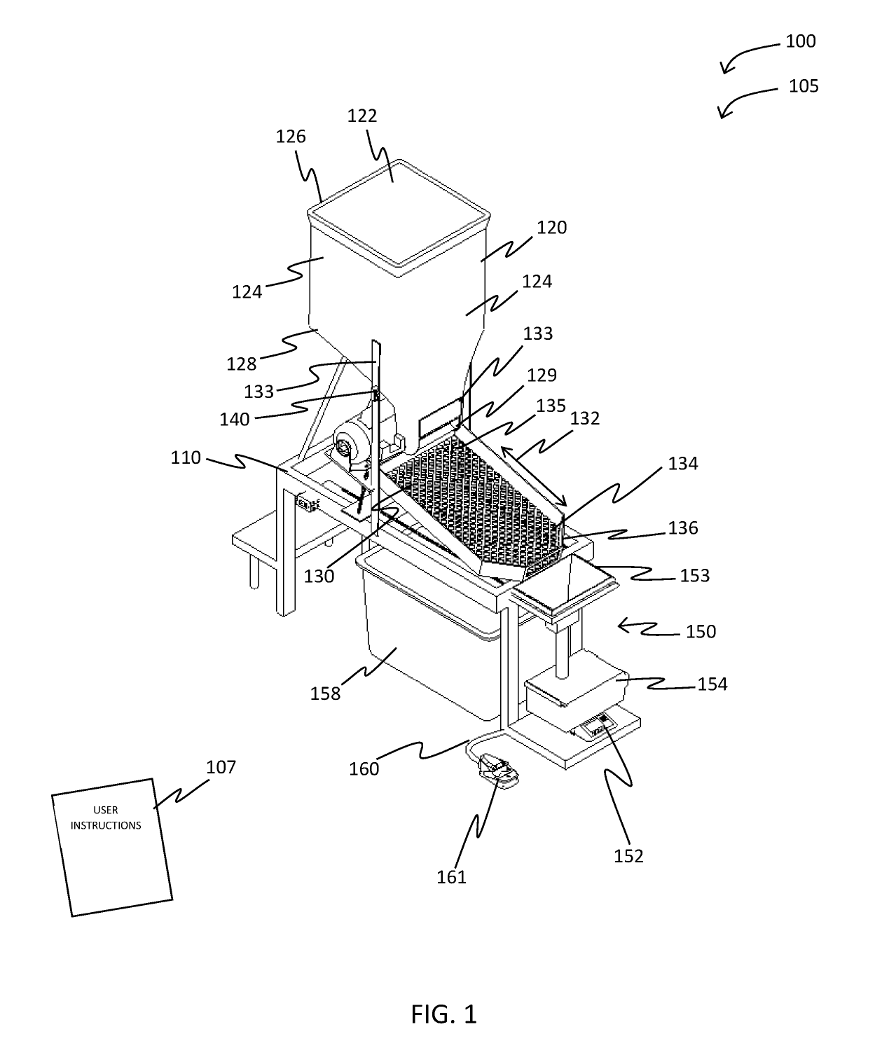

Referring now more specifically to the drawings by numerals of reference, there is shown in FIGS. 1-6, various views of an apparatus for processing cannabis plants 100.

FIG. 1 shows an apparatus for processing cannabis plants (“apparatus” 100) when the cannabis plants each include a plurality of leaves and stems, according to an embodiment of the present disclosure. As illustrated, the apparatus 100 may include a support-frame 110 including a hopper 120, a filter-tray 130, an oscillation means 140, a weighing station 150, and a power supply 160.

As illustrated, the hopper 120 may have an inner-volume 122 defined by at least four-side walls 124, an open-top 126 and a sloped-bottom 128 relative to a floor-surface 5. The inner-volume 122 may be configured to receive at least portions of the cannabis plants, and the sloped-bottom 128 may include an opening 129 configured to discharge the cannabis plants therethrough. In a preferred embodiment, the hopper 120 may be made from a molded fiberglass.

Further illustrated is the filter-tray 130 including a downward-slope 132, at least two support rails 133, and a filter-screen base 134. The downward-slope 132 may have an upward-end 135 and a downward-end 136. Further, the at least two support-rails 133 may be configured to support the filter-tray 130 thereon, and the filter-tray 130 may be configured to receive the cannabis plants discharged from the opening 129 of the hopper 120. In the preferred embodiment, the filter-tray 130 may be made from welded aluminum. Further, the filter-screen base 134 may be made from a punched aluminum sheet. Additionally, an oscillation means 140 may be connected to the filter-tray 130 and configured to oscillate the filter-tray 130 in a forward and backward direction. The oscillation means 140 may be a variable speed motor.

The support-frame 110 may also include a weighing station 150 attached to the downward-end 136 of the filter-tray 130, and the weighing station 150 may include a weighing means 152. The weighing station 150 may be configured to receive the stripped cannabis, and the weighing means 152 may be configured to weigh the stripped cannabis. The apparatus 100 may further comprise a bin 158 located on the floor-surface 5 and underneath the filter-screen base 134, and may be configured to receive the plurality of leaves and stems.

A power supply 160 may also be provided which may be configured to supply power to the oscillation means 140. As shown, the apparatus 100 may further include a power supply pedal 161 configured to selectively turn the power supply 160 to the oscillation means 140 on, and to selectively turn the power supply 160 to the oscillation means 140 off.

According to one embodiment, the apparatus 100 may be arranged as a kit 105. The kit 105 may include a set of instructions 107. The instructions 107 may detail functional relationships in relation to the structure of the apparatus 100 (such that the apparatus 100 can be used, maintained, or the like, in a preferred manner).

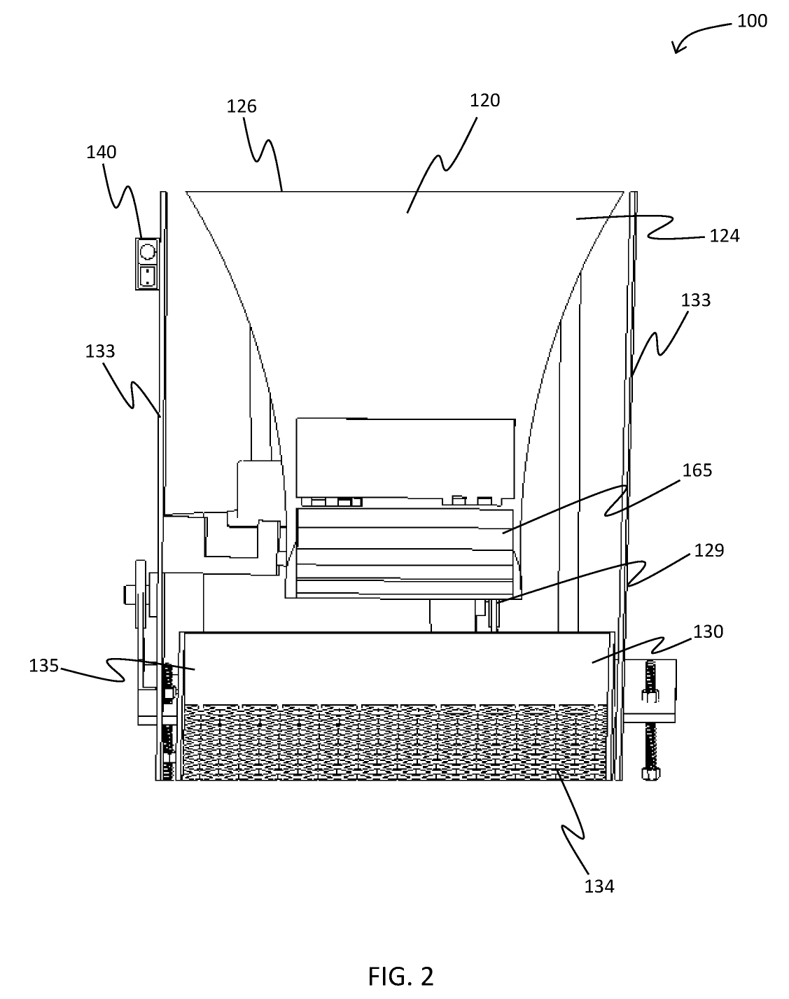

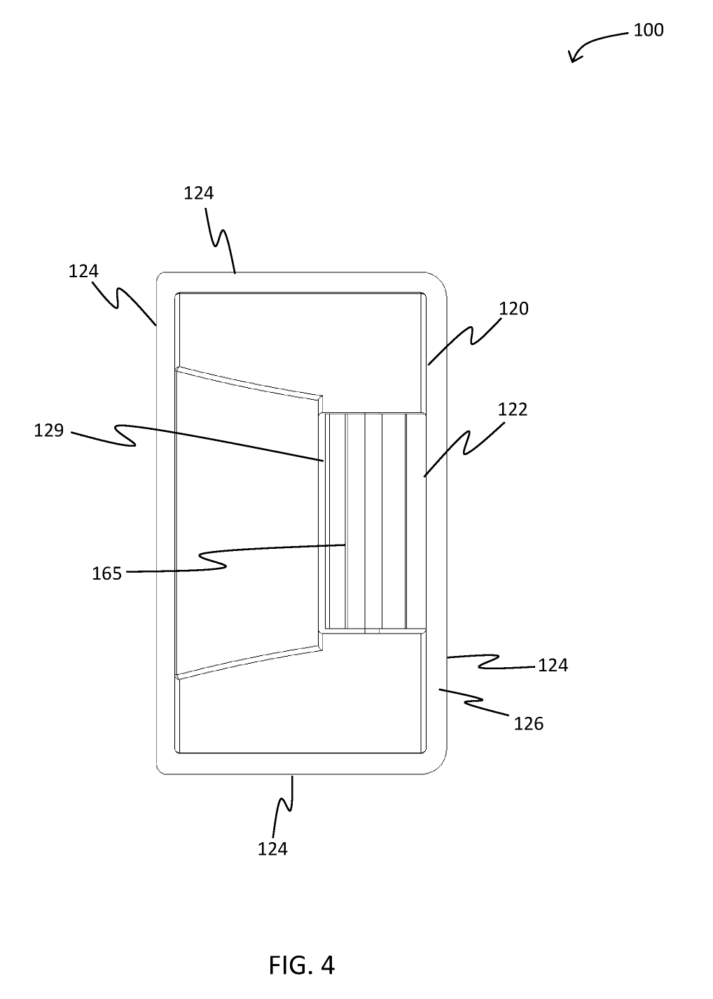

FIG. 2 shows a front perspective view of the apparatus plants 100 of FIG. 1, according to an embodiment of the present disclosure. Shown here is a close-up view of the hopper 120 and the filter-tray 130. Further, as illustrated here, the apparatus 100 may further comprise an auger conveyor 165 located within the inner-volume 122 of the hopper 120 (FIG. 4), the auger conveyor 165 may be configured to aid in transportation of the cannabis plants toward the opening 129 of the hopper 120. In this embodiment, the oscillation means 140 may be further configured to control the auger conveyor 165 (as well as the filter-tray 130).

Here, the oscillation means 140 is shown connected to the filter-tray 130 and as above, the oscillation means 140 may be configured to oscillate the filter-tray 130 in a forward and backward direction. In a preferred embodiment, the oscillation in the forward and backward direction may be configured to strip the cannabis plants of the plurality of leaves and stems, and the oscillation in the forward and backward direction may be further configured to force the plurality of leaves and stems through the filter-screen base 134 of the filter-tray 130. In addition to this, the oscillation in the forward and backward direction and the downward-slope 132 (FIG. 1) may be configured to direct the stripped cannabis plants toward the downward-end 136 of the filter-tray 130. The apparatus 100 may be used after the cannabis plant has flowered and is harvested. The plurality of leaves and stems may be removed from the cannabis plant to reveal the bud of the flower. The plurality of leaves stripped from the cannabis plant may be fan leaves.

Further illustrated is the at least two support-rails 133. In the preferred embodiment, the at least two-support rails 133 may be mounted onto the support-frame 110 via stainless steel thread. In this embodiment, the stainless-steel thread may be configured to allow an angle of the downward-slope 132 to be adjusted. In the preferred embodiment, the at least two support-rails 133 may be made from the welded aluminum. However, it should be appreciated that other materials may be used.

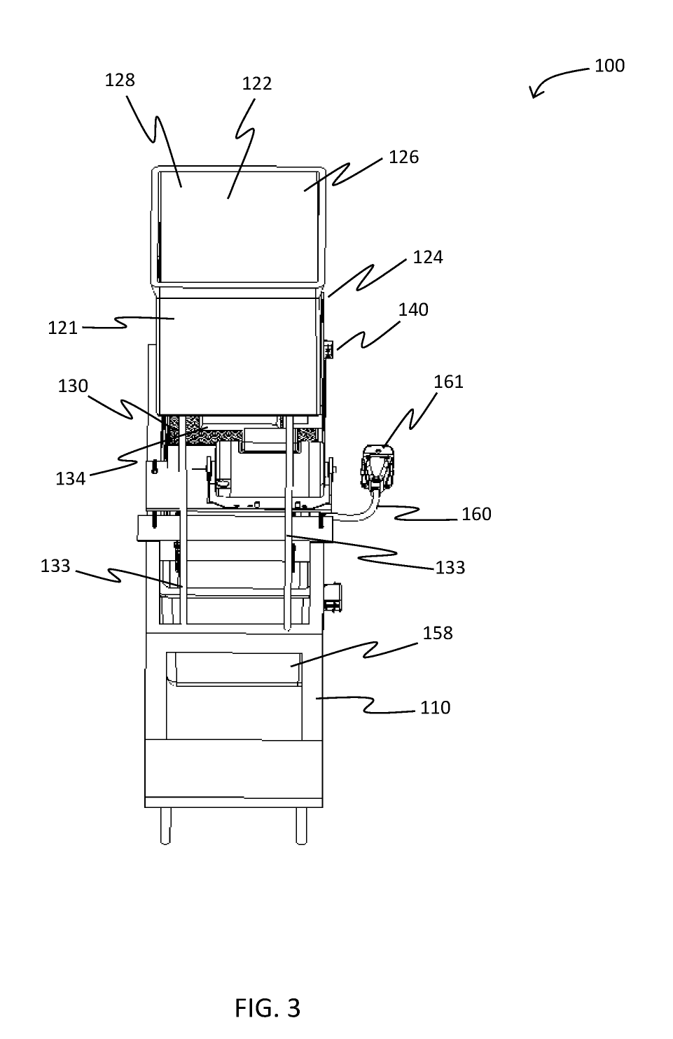

FIG. 3 shows a rear perspective view of the apparatus 100 of FIG. 1, according to an embodiment of the present disclosure. Shown here is the apparatus 100 as shown in FIG. 1 including the support-frame 110 including the hopper 120, the filter-tray 130, the oscillation means 140, the weighing station 150, and the power supply 160.

FIG. 4 shows an aerial perspective view of the apparatus 100 of FIG. 1, according to an embodiment of the present disclosure. Here is shown the auger conveyor 165 located within the inner-volume 122 of the hopper. As above, the auger conveyor 165 may be configured to aid in transportation of the cannabis plants toward the opening 129 of the hopper 120. As illustrated, the auger conveyor 165 may use a rotating helical screw blade to move the cannabis plants. In the preferred embodiment, the auger conveyor 165 may be made from the welded aluminum. However, it should be appreciated that other materials may be used.

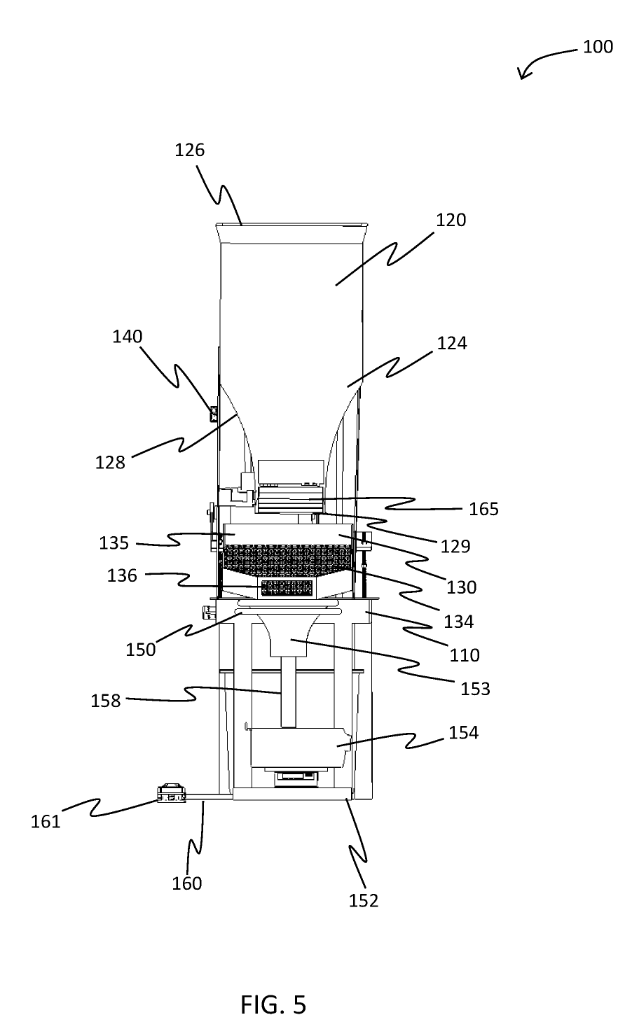

FIG. 5 shows a front perspective view of the apparatus for processing cannabis plants 100 of FIG. 1, according to an embodiment of the present disclosure. As demonstrated, the weighing station 150 may include a funnel 153 and a container 154. In this embodiment, the funnel 153 may configured to funnel 153 a stripped said cannabis plant in to the container 154. In addition, the weighing means 152 may be a digital scale, and the container 154 may be located atop the digital scale. In the preferred embodiment, the weighing station 150 may aid in weighing and packaging the stripped said cannabis plant. In this embodiment, the stripped said cannabis plant may be directed toward the downward-end 136 of the filter-tray 130, into the funnel 153, funneled into the container 154 and weighed by the digital scale. Once weighed, the stripped said cannabis plant in the container 154 may then be packaged for sale.

As above, the apparatus 100 may further comprise the bin 158 located on the floor-surface 5 and underneath the filter-screen base 134, and may be configured to receive the plurality of leaves and stems. The plurality of leaves and stems may be discarded, or alternatively collected to make cannabis products. Examples of cannabis products may include cannabis edibles, skin products, and the like. The present invention may be adjusted to collect the desired end products and to discard the remainder.

Those with ordinary skill in the art will now appreciate that upon reading this specification and by their understanding the art of cannabis harvesting as described herein, methods of using the apparatus 100 for cannabis plant trimming will be understood by those knowledgeable in such art.

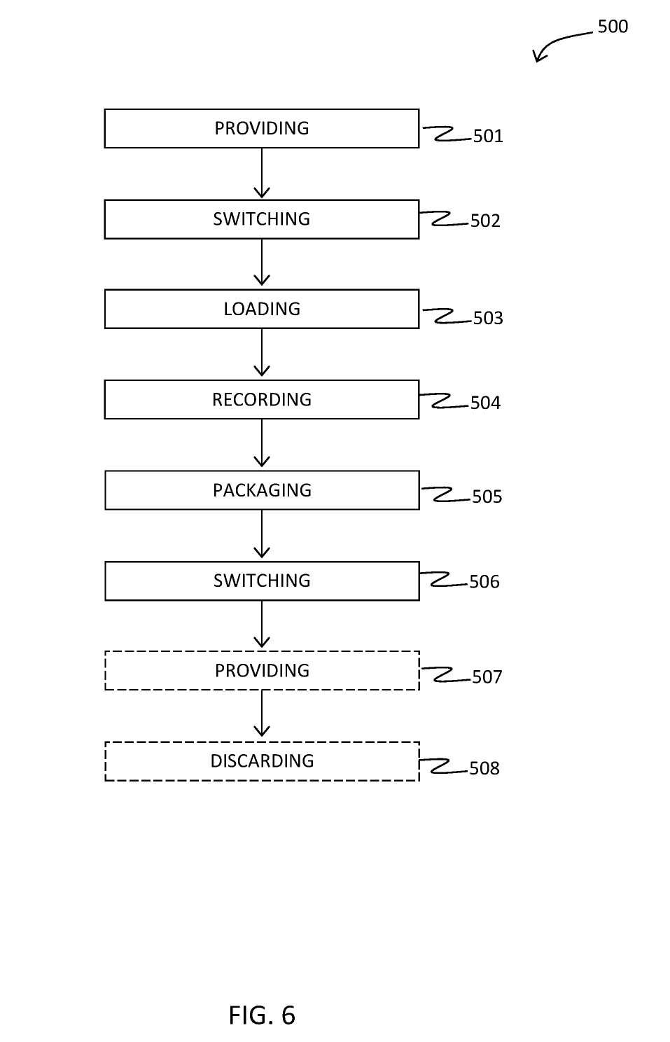

FIG. 6 is a flow diagram 550 illustrating a method for processing cannabis plants 500, according to an embodiment of the present disclosure. As illustrated, the method for processing cannabis plants 500 may include the steps of: step one 501, providing the apparatus 100 as above; step two 502, switching on the power supply 160 to the oscillation means 140; step three 503, loading the cannabis plants into the inner-volume 122 of the hopper 120, step four 504, recording a weight of the stripped cannabis atop the weighing means 152, step five 505, packaging the stripped cannabis; and step six 506, switching off the power supply 160 to the oscillation means 140. Further steps may include: step seven 507, providing the apparatus further including a bin 158 located on the floor-surface 5 and underneath the filter-screen base 134, and configured to receive the plurality of leaves and stems; and step eight 508, discarding the plurality of leaves and stems.

It should be noted that step seven 507 and step eight 508 are optional steps and may not be implemented in all cases. Optional steps of method of use 500 are illustrated using dotted lines in FIG. 6 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for an apparatus for processing cannabis plants 100 (e.g., different step orders within above-mentioned list, elimination or addition of certain steps, including or excluding certain maintenance steps, etc.), are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. An apparatus for processing cannabis plants, the cannabis plants each including a plurality of leaves and stems, the apparatus comprising:
a support-frame, the support-frame including:
a hopper including an inner-volume defined by at least four-side walls, an open-top and a sloped-bottom relative to a floor-surface, the inner-volume being configured to receive at least portions of the cannabis plants, the sloped-bottom including an opening configured to discharge the cannabis plants therethrough;
a filter-tray including a downward-slope, at least two support rails, and a filter-screen base, the downward-slope having an upward-end and a downward-end, the at least two support-rails configured to support the filter-tray thereon, and the filter-tray being configured to receive the cannabis plants discharged from the opening of the hopper;
an oscillation means connected to the filter-tray, the oscillation means configured to oscillate the filter-tray in a forward and backward direction; and
wherein the oscillation in the forward and backward direction is configured to strip the cannabis plants of the plurality of leaves and stems; and
wherein the oscillation in the forward and backward direction is further configured to force the plurality of leaves and stems through the filter-screen base of the filter-tray; and
wherein the oscillation in the forward and backward direction and the downward-slope are configured to direct the stripped cannabis plants toward the downward-end of the filter-tray; and
a weighing station attached to the downward-end of the filter-tray, the weighing station including a weighing means, the weighing station being configured to receive the stripped cannabis, and the weighing means being configured to weigh the stripped cannabis; and
a power supply configured to supply power to the oscillation means.

2. The apparatus of claim 1, further comprising an auger conveyor located within the inner-volume of the hopper, the auger conveyor configured to aid in transportation of the cannabis plants toward the opening of the hopper.

3. The apparatus of claim 1, wherein the at least two-support rails are mounted onto the support-frame via stainless steel thread.

4. The apparatus of claim 3, wherein the stainless-steel thread is configured to allow an angle of the downward-slope to be adjusted.

5. The apparatus of claim 1, wherein the oscillation means is a variable speed motor.

6. The apparatus of claim 5, further comprising the auger conveyor, and wherein the oscillation means is further configured to control the auger conveyor.

7. The apparatus of claim 1, further comprising a power supply pedal configured to selectively turn the power supply to the oscillation means on, and to selectively turn the power supply to the oscillation means off.

8. The apparatus of claim 1, wherein the weighing station includes a funnel and a container, and wherein the funnel is configured to funnel a stripped said cannabis plant in to the container.

9. The apparatus of claim 8, wherein the weighing means is a digital scale.

10. The apparatus of claim 9, wherein the container is located atop the digital scale.

11. The apparatus of claim 1, further comprising a bin located on the floor-surface and underneath the filter-screen base, and configured to receive the plurality of leaves and stems.

12. The apparatus of claim 1, wherein the hopper is made from a molded fiberglass.

13. The apparatus of claim 1, wherein the auger conveyor is made from welded aluminum.

14. The apparatus of claim 13, wherein the at least two support-rails are made from the welded aluminum.

15. The apparatus of claim 13, wherein the filter-tray is made from the welded aluminum.

16. The apparatus of claim 1, wherein the filter-screen base is made from a punched aluminum sheet.

17. An apparatus for processing cannabis plants, the cannabis plants each including a plurality of leaves and stems, the apparatus comprising:
a support-frame, the support-frame including:
a hopper including an inner-volume defined by at least four-side walls, an open-top and a sloped-bottom relative to a floor-surface, the inner-volume being configured to receive at least portions of the cannabis plants, the sloped-bottom including an opening configured to discharge the cannabis plants therethrough;
a filter-tray including a downward-slope, at least two support rails, and a filter-screen base, the downward-slope having an upward-end and a downward-end, the at least two support-rails configured to support the filter-tray thereon, and the filter-tray being configured to receive the cannabis plants discharged from the opening of the hopper;
an oscillation means connected to the filter-tray, the oscillation means configured to oscillate the filter-tray in a forward and backward direction; and
wherein the oscillation in the forward and backward direction is configured to strip the cannabis plants of the plurality of leaves and stems; and
wherein the oscillation in the forward and backward direction is further configured to force the plurality of leaves and stems through the filter-screen base of the filter-tray; and
wherein the oscillation in the forward and backward direction and the downward-slope are configured to direct the stripped cannabis plants toward the downward-end of the filter-tray; and
a weighing station attached to the downward-end of the filter-tray, the weighing station including a weighing means, the weighing station being configured to receive the stripped cannabis, and the weighing means being configured to weigh the stripped cannabis; and
a power supply configured to supply power to the oscillation means; and
wherein the apparatus further comprises an auger conveyor located within the inner-volume of the hopper, the auger conveyor configured to aid in transportation of the cannabis plants toward the opening of the hopper;
wherein the at least two-support rails are mounted onto the support-frame via stainless steel thread;
wherein the stainless-steel thread is configured to allow an angle of the downward-slope to be adjusted;
wherein the oscillation means is a variable speed motor;
wherein the oscillation means is further configured to control the auger conveyor;
wherein the apparatus further comprises a power supply pedal configured to selectively turn the power supply to the oscillation means on, and to selectively turn the power supply to the oscillation means off;
wherein the weighing station includes a funnel and a container, and
wherein the funnel is configured to funnel a stripped said cannabis plant in to the container;
wherein the weighing means is a digital scale;
wherein the container is located atop the digital scale;
wherein the apparatus further comprises a bin located on the floor-surface and underneath the filter-screen base, and configured to receive the plurality of leaves and stems;
wherein the hopper is made from a molded fiberglass;
wherein the auger conveyor is made from welded aluminum;
wherein the at least two support-rails are made from the welded aluminum;
wherein the filter-tray is made from the welded aluminum; and
wherein the filter-screen base is made from a punched aluminum sheet.

18. The apparatus of claim 17, further comprising set of instructions; and
wherein the apparatus is arranged as a kit.

19. A method for processing cannabis plants, the cannabis plants including a plurality of leaves and stems, the method comprising:
providing an apparatus for processing cannabis plants, the apparatus including:
a support-frame, the support-frame including:
a hopper including an inner-volume defined by at least four-side walls, an open-top and a sloped-bottom relative to a floor-surface, the inner-volume being configured to receive at least portions of the cannabis plants, the sloped-bottom including an opening configured to discharge the cannabis plants therethrough;
a filter-tray including a downward-slope, at least two support rails, and a filter-screen base, the downward-slope having an upward-end and a downward-end, the at least two support-rails configured to support the filter-tray thereon, and the filter-tray being configured to receive the cannabis plants discharged from the opening of the hopper;
an oscillation means connected to the filter-tray, the oscillation means configured to oscillate the filter-tray in a forward and backward direction; and
wherein the oscillation in the forward and backward direction is configured to strip the cannabis plants of the plurality of leaves and stems; and
wherein the oscillation in the forward and backward direction is further configured to force the plurality of leaves and stems through the filter-screen base of the filter-tray; and
wherein the oscillation in the forward and backward direction and the downward-slope are configured to direct the stripped cannabis plants toward the downward-end of the filter-tray; and
a weighing station attached to the downward-end of the filter-tray, the weighing station including a weighing means, the weighing station being configured to receive the stripped cannabis, and the weighing means being configured to weigh the stripped cannabis; and
a power supply configured to supply power to the oscillation means;
switching on the power supply to the oscillation means;
loading the cannabis plants into the inner-volume of the hopper;
recording a weigh of the stripped cannabis atop the weighing means;
packaging the stripped cannabis; and
switching off the power supply to the oscillation means.

20. The method of claim 19, further comprising the steps of:
providing the apparatus further including:
a bin located on the floor-surface and underneath the filter-screen base, and configured to receive the plurality of leaves and stems; and
discarding the plurality of leaves and stems.
